(19) United States
(12) Patent Application Publication
(10) Pub. No.: US 2025/0242107 A1
(43) Pub. Date: Jul. 31, 2025

(54) MEDICAL PERISTALTIC PUMP

(71) Applicant: B. Braun Melsungen AG, Melsungen (DE)

(72) Inventors: Mario Richardt, Zierenberg (DE); Wilhelm Schwendich, Petersberg (DE); Matthias Schwalm, Schwalmstadt (DE)

(21) Appl. No.: 19/039,042

(22) Filed: Jan. 28, 2025

(30) Foreign Application Priority Data
Jan. 29, 2024 (DE) 10 2024 102 479.8

(51) Int. Cl. A61M 5/142

(52) U.S. Cl. CPC A61M 5/14228; A61M 2205/106

(57) ABSTRACT

A medical peristaltic pump, in particular for medical dosing, includes a tube holder, a clamping mechanism and a locking lever. A tube can be inserted into the tube holder and fixed. The clamping mechanism has a first clamping jaw that is prestressed against a second clamping jaw into a clamped position in which the tube can be clamped. The first clamping jaw can be actuated from the clamped position into an open position, in which the tube can be released and/or inserted. The locking lever can be clamped, in particular tensioned, indirectly or directly against the first clamping jaw by at least one spring element so that the first clamping jaw is supported indirectly or directly on the locking lever in the open position.

### CROSS-REFERENCE TO RELATED APPLICATION

This application claims priority under 35 U.S.C. § 119 to German Application No. 10 2024 102 479.8, filed on Jan. 29, 2024, the content of which is incorporated by reference herein in its entirety.

### FIELD

This disclosure concerns a medical peristaltic pump.

### BACKGROUND

A peristaltic pump can be used to dose medical agents or pump blood in extracorporeal blood treatment. One possible mode of action of such a peristaltic pump is to impress a peristaltic movement on a tube through which a fluid is passed, by means of a displacement unit of the peristaltic pump, whereby the fluid is conveyed from a container of an active agent to an access point on the patient.

A peristaltic pump of radial design is disclosed in the patent EP 3 061 473 B1 of the applicant. The peristaltic pump shown for extracorporeal blood treatment has a motorized rotary drive with a rotor on which radially outward squeezing elements are provided, which during rotation repeatedly slide or roll off a tube section fixed to the housing and supported in an arc in a longitudinal direction of the tube.

A peristaltic pump of linear design is shown in the patent EP 0 484 717 B1 of the applicant. It has a tube holder for a tube through which fluid is conveyed, as well as a displacement unit with a multitude of slide-like displacers, which are arranged next to one another along the tube holder and are guided in a shaft-like housing section so that they can move in a linear fashion at right angles to the holder. The tube is inserted into the tube holder and a housing flap is closed so that the tube is fixed in the tube holder by the housing flap. When the housing flap is closed, the time-delayed actuated displacers work against the tube, causing it to be pinched at the housing flap and to move in a peristaltic pattern, which in turn causes the fluid to be conveyed.

A redundant clamping system is common to ensure that fluid only flows to the patient when needed, i.e., only when the peristaltic pump is operating, and not, for example, when the tube is inserted into the peristaltic pump. On the one hand, a tube clamp is provided on the tube itself for this purpose, and on the other hand, the peristaltic pump has its own clamp mechanism, the clamp of which is activated when the tube is inserted and the housing flap is closed.

Peristaltic pumps of this type are known from the applicant's range under the registered trademarks INFUSOMAT® SPACE and INFUSOMAT® SPACE^{PLUS}, whose clamp has one fixed and one movable clamping jaw. The movable clamping jaw is coupled to a pressure lever that can be felt by the user and can be actuated by the user to an open position in which the clamp is open and the tube can be inserted or removed. In this open position, the movable clamping jaw is locked and fixed with a locking lever of the clamping mechanism. The locking lever is in turn indirectly tensioned against the movable clamping jaw by a spring element designed as a spiral spring, so that the latching and thus the open position of the clamp is secured.

The locking lever and spiral spring are small in size, in the millimeter range, making assembly difficult and prone to error. In addition, the locking lever performs a rotational movement, whereas the spiral spring, designed as a pressure spring, is more suitable for linear movements.

### SUMMARY

The purpose of the present disclosure is to provide a medical peristaltic pump that reduces or eliminates the disadvantages of the state of the art and that is, in particular, simplified in terms of design compared to the state of the art.

The problem is solved by a medical peristaltic pump, in particular for medical dosing, in particular for dosing active ingredients or for nutrition, or for pumping blood, in particular in an extracorporeal blood circuit. The peristaltic pump can be a component of a dialysis machine, an infusion pump, or a syringe pump. According to the disclosure, the peristaltic pump has a tube holder that is designed and configured to allow a tube, in particular an infusion tube, to be inserted into it and fixed in place. The peristaltic pump according to the disclosure also has a clamp mechanism with a first clamping jaw that is prestressed against a second clamping jaw in a clamped position in which an inserted tube can be clamped, in particular is clamped. From the clamped position, the first clamping jaw can be actuated into its open position, in which the tube can be released, in particular released, and/or inserted. Furthermore, the clamping mechanism has a locking lever which can be clamped, in particular tensioned, directly or indirectly against the first clamping jaw by at least one spring element, so that the first clamping jaw is supported directly or indirectly on the locking lever in the open position, in particular in such a way that it is locked in the open position. According to the disclosure, the locking lever is formed integrally with the at least one spring element, in particular in a single piece of material.

The advantage of the one-piece design is that the number of parts has been reduced from two to one, eliminating the need to align, position, and assemble two small components. This simplifies the assembly considerably and makes it less prone to errors.

In one possible design, the tube holder consists of a tube compartment and a tube compartment cover that closes the tube compartment. The tube can be placed in the tube compartment when the tube compartment cover is open and can be fixed, in particular firmly fixed, by closing the tube compartment cover.

According to a preferred further embodiment, the at least one spring element of the locking lever is designed as a spring tab and, in particular, is formed on a base body of the locking lever.

Since medical peristaltic pumps require a high degree of availability and reliability and the safe insertion and clamping, as well as release of a tube, are a basic requirement for operation, the locking lever, which is formed in one piece from the base body and spring tab, is designed for tens of thousands of operations.

In principle, a rotating bearing of the base body on the housing side has proven to be advantageous. A spring effect is achieved in a simple way by angling the at least one spring tab against the base body in the plane of rotation, which is then further developed. In this way, both the at least one spring tab and the base body form a kind of V-shape in the plane of rotation.

According to a preferred embodiment, the at least one spring tab is supported on the housing side and on the other hand it indirectly tensions the base body of the locking lever—in particular via a pressure lever that can be felt by a user, to which the movable clamping jaw is coupled and via which the movable clamping jaw can be actuated/felt into an open position or directly against the movable clamping jaw.

The at least one spring tab has a particularly robust and simple shape according to a further embodiment in which it is curved, in particular curved in an S-shape configuration, in particular attached to the base body.

The S-shaped design/connection ensures power transmission and durability during movements of the movable clamping jaw into the open and clamped positions and represents a form of spring tab that is optimized for power flow.

It is particularly advantageous with regard to the introduction of force from the at least one spring tab into the base body if, in accordance with a further embodiment, a direction, with which the at least one spring tab is attached to the base body, in particular molded on, intersects an axis of rotation of the locking lever.

Preferably, the at least one spring tab is tied approximately or exactly at a right angle, in particular molded on.

The above-mentioned V-shape may have an included angle of less than 90°. The opening angle is measured in particular with respect to the axis of rotation of the locking lever between an end section of the at least one spring tab, with which the at least one spring tab can be/is supported on the housing side, and an end section of the base body, with which the base body can be/is indirectly or directly clamped against the movable clamping jaw.

In particular, the end section of the at least one spring tab, which is attached at a right angle, is bent at an angle of less than 90° towards the base body.

According to a further embodiment, an end section of the at least one spring tab is supported on the housing side with a line contact, which extends preferably as normal to the plane of rotation, or with a surface contact. The line contact allows the end section of the at least one spring tab to slide smoothly on the housing-side component or a housing-side support surface while still providing good protection against wear.

According to a further development, in particular in order to realize the last-mentioned line contact, an end section with which the at least one spring tab is supported on the housing side has a convexly curved, in particular partially cylindrical, outer lateral surface towards the support surface on the housing side.

Alternatively or in addition, the end section with which the at least one spring tab is supported on the housing side is bent. The curved end section allows the bearing to slide freely on a housing-side contact or support surface. In particular, no further formalized management is necessary for this.

According to the further embodiment, the width of the at least one spring tab is designed to prevent or at least minimize the at least one spring tab from tipping out of the plane of rotation. Preferably, the width of the at least one spring tab, in particular measured in a direction perpendicular to the plane of rotation, is at least two to about ten times the thickness of the at least one spring tab, in particular measured in the plane of rotation.

According to a preferred design, the base body has a preferably cylindrical bearing section that is rotatably mounted on the housing side. The bearing section is followed, preferably in the direction of the axis of rotation, by a connecting section that tapers towards the bearing section, to which the at least one spring tab is attached.

In another embodiment, both the bearing section and the connecting section are cylindrical, and in particular of the same diameter.

The at least one spring tab is preferably molded/attached to the connecting section with a line contact. The term “line contact” is to be understood as meaning that a cross-section of the molding/connecting is linear. The at least one spring tab is attached to the connecting section in this way, similar to a hinge.

To ensure a certain amount of leeway for the at least one spring tab in relation to the bearing section, a slot or gap is provided between the at least one spring tab and the bearing section.

According to a further embodiment, with which a spring travel of the base body relative to the at least one spring tab can be influenced, at least one leg extends away from the base body and/or from the at least one spring tab, forming a travel limiter or a stop with regard to compression and/or extension of the at least one spring tab, or forming a force support.

In order to increase a spring force or to adjust it better depending on the distance and/or to be able to control the application of force to the clamping jaw better, in particular in different directions of movement, force, and/or compression/rebound, several spring tabs are attached to the base body in accordance with a further development, which extend in particular in different directions of movement, force, and/or compression/rebound.

In particular, at least one spring tab is provided that supports or dampens movement of the movable clamping jaw into the open position, and another that is effective in the direction of the clamped position of the movable clamping jaw.

### BRIEF DESCRIPTION OF THE DRAWINGS

The disclosure is explained in more detail below with reference to two exemplary, non-limiting embodiments shown in the figures.

FIG. 1 a medical peristaltic pump according to one exemplary embodiment in a front view;

FIG. 2 the peristaltic pump according to FIG. 1 with the front flap open;

FIG. 3 a clamping mechanism of the medical peristaltic pump according to FIGS. 1 and 2 in a perspective view;

FIG. 4 a locking lever according to a first exemplary embodiment, which is installed in the clamping mechanism according to FIG. 3, in a perspective view;

FIG. 5 the locking lever as seen from below, in accordance with FIG. 4;

FIG. 6 the locking lever as shown in FIGS. 4 and 5, in a side view; and

FIG. 7 a locking lever according to a second exemplary embodiment, which can be installed in the clamping mechanism according to FIG. 3.

### DETAILED DESCRIPTION

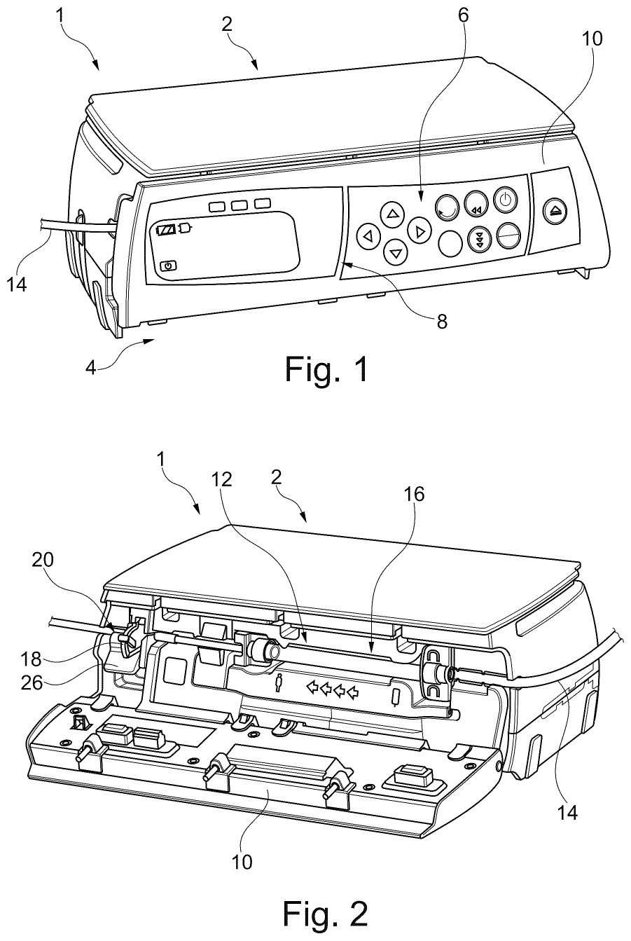

FIG. 1 shows a medical peristaltic pump 1 designed as an infusion device in a front view in perspective. The infusion device, or rather the peristaltic pump 1, has a substantially cuboid housing 2 with a front side 4 that is formed by a foldable tube compartment cover 10 hinged on the underside, on which a control panel 6 and a display 8 are arranged. The tube compartment cover 10 closes one tube compartment 12, into which an infusion tube 14 is placed (see also FIG. 2).

FIG. 2 shows the infusion device 1 as shown in FIG. 1 in a front view from a perspective view, with open tube compartment 12, i.e., with the tube compartment cover 10 flipped up. In FIG. 2, starting from the right, an infusion container (not shown) extends into the infusion tube 14 in the tube compartment 12. The infusion tube 14 is inserted as intended.

The tube compartment 12, together with the tube compartment cover 10, forms a tube holder according to the disclosure, which is designed so that the infusion tube 14 can be inserted into it and—in the illustrated exemplary embodiment—can be fixed in place by closing the tube compartment cover 10.

The tube compartment 12 has a tube inlet 20 in the area of what is, from an operating point of view, the right-hand side wall, as well as a tube outlet in the area of a left-hand side wall. The folded-down tube compartment cover 10, as shown in FIG. 2, together with the bottom or base of the tube compartment 12, forms mold sections into which the infusion tube 14 is inserted in its operating position. The infusion tube 14 is fixed in this operating position by closing the tube compartment cover 10.

With reference to the direction of conveyance from right to left as shown in FIG. 2, on the right at the bottom of the tube compartment 12 there is an essentially rectangular recess 16 which is spanned by a membrane, behind which are arranged linear displacers, which, during operation, squeeze the infusion tube 14 in a peristaltic sequence against the closed tube compartment cover 10 and thus convey fluid from right to left to the patient.

A redundant clamping system is provided to ensure that the fluid flows to the patient only via peristaltic pump 1 and not prematurely, for example, when the infusion tube 14 is inserted into the tube compartment 12. On the one hand, a tube clamp 18 is already provided on the infusion tube 14 itself for this purpose, and on the other hand, the peristaltic pump 1 in the housing 2 has a clamping mechanism that is largely located inside the housing 2 and is therefore not shown in FIG. 2.

However, for better orientation, FIG. 2 indicates a position of the clamp mechanism with its reference sign 20, from which it can be seen that it is arranged in close spatial proximity to the inserted tube clamp 18. The clamp mechanism 20 is activated when the infusion tube 14 is inserted by inserting the tube clamp 18 into the clamp mechanism 20 and then closing the tube compartment cover 10.

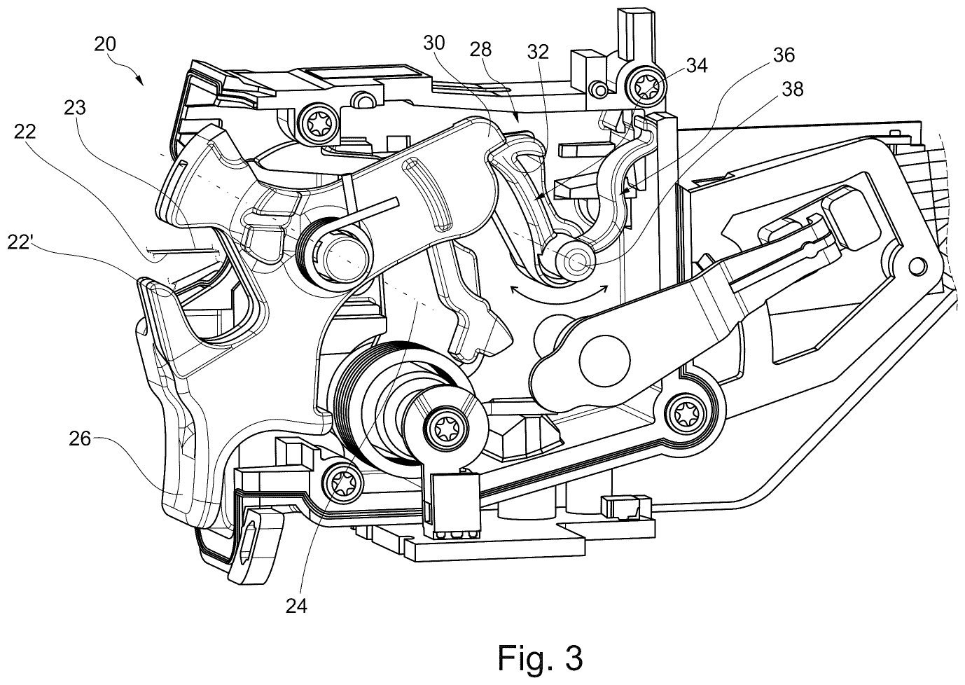

FIG. 3 shows the clamping mechanism 20 in a side view, which, according to FIG. 2, corresponds approximately to a viewing direction from right to left. The clamping mechanism 20 has two clamping jaws 22 and 23, of which only clamping sections are shown schematically in FIG. 3 in the clamped position. In the illustrated clamped position (22, 23), the clamping sections have a small, predetermined gap, which corresponds approximately to twice the wall thickness of the infusion tube 14, so that the tube is completely clamped in the clamped position but is not damaged. The second clamping jaw 23 is fixed.

The first clamping jaw 22 can be rotated around an axis of rotation 24, i.e., it is movable. The first clamping jaw 22 is coupled to a pressure lever 26 that can be felt by a user (see also FIG. 2), via which the clamping jaw 22 can be actuated into an open position shown in FIG. 3, represented by the reference sign 22′, in which the first clamping jaw 22′ is open and offers, with the second clamping jaw 23, a mouth-like opening into which the infusion tube 14 can be inserted. (See FIG. 2, infusion tube 14 inserted here, clamped position).

In the open position as shown in FIG. 3, the movable clamping jaw 22′, which has been actuated into the open position, is latched in a manner that is obvious in the manner shown indirectly, in the exemplary embodiment shown via the pressure lever 26, with a locking lever 28 of the clamping mechanism 20, and in this way fixed in its open position. The latching is realized by a latching cam 30 of the pressure lever 26 resting on a latching section 32 of the locking lever 28.

The locking lever 28 is apparently formed by a base body 34 having the latching section 32 and a spring tab 36. The latter is apparently formed integrally with the base body 34. For this purpose, the at least one spring tab 36 is molded onto the base body 34. The at least one spring tab 36 extends from the base body 34 in an S-shaped curve. The locking lever 28 is rotatably mounted around an axis of rotation 38 and has a substantially V-shaped basic shape in a plane of rotation.

The at least one spring tab 36 is supported on the housing side by an end section that has been removed from the base body 34 and is prestressed. In this way, the base body 34 is clamped against the pressure lever 26, which indirectly secures the first clamping jaw 22′ in its open position.

When the infusion tube 14 is inserted into the peristaltic pump 1, the tube clamp 18 is pushed into its associated shaft in the clamp mechanism 20 (see FIG. 2) and actuates the locking lever 28 inside the housing 2 by moving its base body 34 indirectly (via an intermediate lever not shown) and, among other things, against the spring force of the at least one spring tab 36, clockwise about the axis of rotation 38 from the open position/latched position shown in FIG. 3. As a result, the pressure lever 26 snaps clockwise about the axis of rotation 24 due to its prestressing and takes the first movable clamping jaw 22′, also clockwise, into its clamped position (represented by the reference sign 22) and the clamping mechanism 20 clamps the infusion tube 14 downstream of the tube clamp 18 (see position of the pressure section 26, FIG. 2).

When the tube compartment cover 10 is closed, it pushes the tube clamp 18, which is designed as a sliding clamp, into its open position, following the previously described closing of the clamping mechanism 20.

In this way, the clamping function of the clamp 18 was automatically transferred to the clamping function of the clamping mechanism—by inserting the infusion tube 14 and closing the tube compartment cover 10. Fluid flow does not occur until a user enters the appropriate command at the control panel 6.

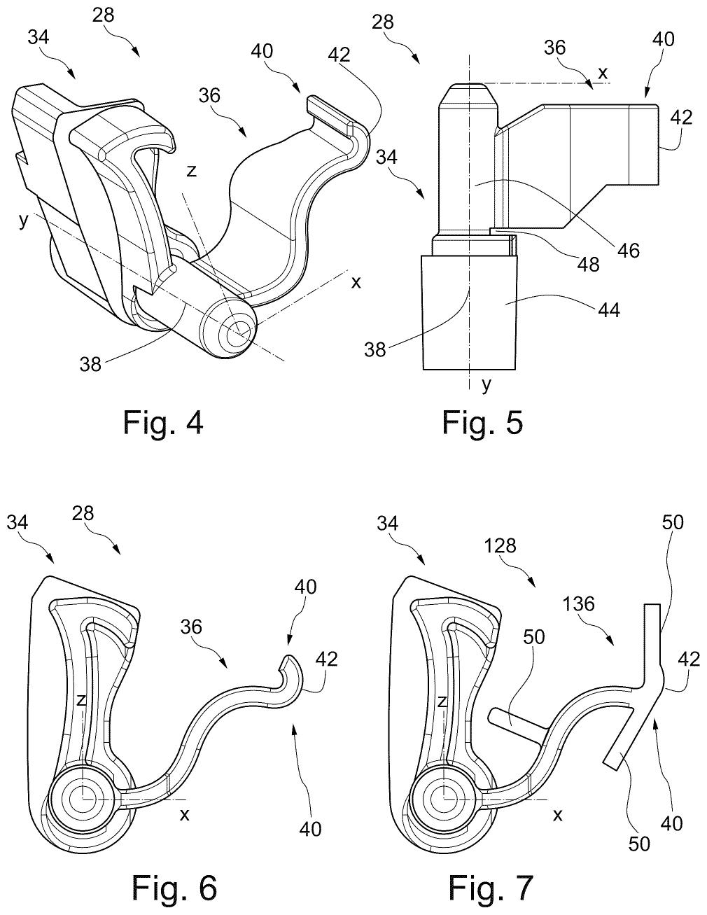

FIG. 4 shows the locking lever 28 in a perspective view according to FIG. 3, in a separate illustration. Here it can be clearly seen that an end section 40, with which the at least one spring tab 36 is supported on the housing side (see FIG. 3), has a partially cylindrical outer lateral surface 42 that is convexly curved towards the support surface on the housing side, by means of which the at least one spring tab can slide off easily. According to FIG. 4, a slot 48 is provided between the at least one spring tab 36 and the bearing section 44 in order to ensure that the at least one spring tab 36 has a leeway relative to the bearing section 44.

FIG. 5 shows the released locking lever 28 according to FIG. 4 in a view from below and clarifies, in conjunction with FIGS. 4 and 6, that a width of the at least one spring tab 36, measured in a direction perpendicular to the plane of rotation, which in the figures is the direction of the axis of rotation 38 (also designated as y), is approximately five to ten times the thickness of the at least one spring tab 36, wherein the thickness is measured in the rotation plane, that is to say in the x-z plane according to FIG. 4 or FIG. 6. Preferably, this width is at least twice the thickness of the at least one spring tab 36. In this way, the at least one spring tab 36 has a high torsional stiffness, so that lateral buckling and/or yielding of the at least one spring tab 36 is prevented, especially during compression.

FIG. 6 shows the released locking lever 28 according to FIG. 4 or 5 in a side view, wherein in particular the S-shaped curved form of the at least one spring tab 36 is illustrated.

FIG. 7 shows a locking lever 128 according to a second exemplary embodiment, which can also be installed in the clamping mechanism 20 according to FIG. 3, in a side view. In a departure from the exemplary embodiment according to FIGS. 3 through 6, several legs 50 extend away from the at least one spring tab 36, thereby forming a travel limiter or a stop with regard to the springing of the at least one spring tab 36 against the base body 34 and the springing out of the at least one spring tab 36, as well as an additional force support for the at least one spring tab 36 in its end positions.

### REFERENCE CHARACTER LIST

1 peristaltic pump
2 housing
4 front side
6 control panel
8 display
10 tube compartment cover
12 tube compartment
14 infusion tube
16 recess
18 tube clamp
20 clamping mechanism
22 first clamping jaw (clamped position)
22′ first clamping jaw (open position)
23 second clamping jaw
24 axis of rotation
26 pressure lever
28; 128 locking lever
30 latching cam
32 latching section
34 base body
36; 136 spring tab
38 axis of rotation
40 end section
42 outer lateral surface
44 bearing section
46 connecting section
48 slot
50 leg

1. A medical peristaltic pump comprising:
a tube holder configured to receive a tube in a fixed position;
a clamp comprising a first clamping jaw and a second clamping jaw, and
a locking lever controlled by at least one spring element,
the first clamping jaw being prestressed against the second clamping jaw into a clamped position in which the tube is clampable,
the first clamping jaw configured to be actuated from the clamped position into an open position, in which the tube is releasable and/or inserted,
the locking lever configured to be clamped directly or indirectly against the first clamping jaw so that the first clamping jaw is supported indirectly or directly on the locking lever in the open position.

2. The medical peristaltic pump according to claim 1, wherein the locking lever and the at least one spring element are formed as a one-piece homogenous body of unitary construction.

3. The medical peristaltic pump according to claim 1 further comprising a housing, wherein the at least one spring element comprises at least one spring tab integrally formed on a base body of the locking lever.

4. The medical peristaltic pump according to claim 3, wherein:
the base body is rotatably mounted on the housing,
the at least one spring tab is angled in a plane of rotation against the base body,
the at least one spring tab is supported on the housing, and
the base body is clamped indirectly or directly against the first clamping jaw by the at least one spring tab.

5. The medical peristaltic pump according to claim 3, wherein the at least one spring tab is curved.

6. The medical peristaltic pump according to claim 5, wherein the at least one spring tab has an S-shaped configuration.

7. The medical peristaltic pump according to claim 3, wherein one direction, with which the at least one spring tab is integrally formed on the base body, intersects an axis of rotation of the locking lever.

8. The medical peristaltic pump according to claim 3, wherein an end section, with which the at least one spring tab is supported on the housing, has an outer lateral surface that is convexly curved, towards a support surface on the housing.

9. The medical peristaltic pump according to claim 3, wherein the at least one spring tab has a width that is at least 2 to 10 times a thickness of the at least one spring tab.

10. The medical peristaltic pump according to claim 9, wherein the width of the at least one spring tab is measured in a direction of an axis of rotation of the locking lever and perpendicular to a plane of rotation of the at least one spring tab, and the thickness of the at least one spring tab is measured in the plane of rotation.

11. The medical peristaltic pump according to claim 3, wherein the base body has a bearing section that is rotatably mounted on the housing and a connecting section that tapers towards the bearing section to which the at least one spring tab is connected.

12. The medical peristaltic pump according to claim 11, wherein a slot or gap is provided between the at least one spring tab and the bearing section.

13. The medical peristaltic pump according to claim 3, further comprising at least one leg that extends away from the base body and/or from the at least one spring tab.

14. The medical peristaltic pump according to claim 13, wherein the at least one leg forms a travel limiter or a stop with respect to compression and/or rebound of the at least one spring tab.

15. The medical peristaltic pump according to claim 13, wherein the at least one leg forms a force support.

16. The medical peristaltic pump according to claim 3, wherein the at least one spring tab supports or dampens movement of the first clamping jaw into the open position.

17. The medical peristaltic pump according to claim 3, wherein the at least one spring tab is effective in a direction of the clamped position.
